(19) United States
(12) Patent Application Publication
(10) Pub. No.: US 2021/0361188 A1
(43) Pub. Date: Nov. 25, 2021

(54) POSTURE SUPPORT DEVICE WITH ADDITIONAL FUNCTIONS

(71) Applicant: Poras Patel, South Plainfield, NJ (US)

(72) Inventor: Poras Patel, South Plainfield, NJ (US)

(21) Appl. No.: 16/882,465

(22) Filed: May 23, 2020

(51) Int. Cl. A61B 5/11; A61B 5/00; A61J 1/03

(52) U.S. Cl. CPC A61B 5/1116; A61B 5/747; A61B 5/7405; A61B 2562/02; A61B 5/742; A61J 1/03; A61B 2560/0431; A61B 5/0022

(57) ABSTRACT

A posture support device, including a main body to support a weight of a user thereon, a control unit disposed within at least a portion of the main body to connect the user to an external mobile device of a third party in response to an emergency, and to display the third party on the control unit, a speaker disposed on at least a portion of the main body to emit at least one sound received from the external mobile device during conversations with the third party, and a medication compartment disposed around at least a portion of the main body to store at least one medication therein.

### BACKGROUND

### 1. Field

The present general inventive concept relates generally to posture support, and particularly, to a posture support device with additional functions.

### 2. Description of the Related Art

Falling is a major problem among elderly persons due to age-related fragility of the body. When an elderly person falls, he or she often needs immediate medical care. Unfortunately, the elderly person's need of urgent care is often unmet or provided late resulting in an aggravated injury or death.

A conventional cane or walking stick is a common solution for elderly persons that need support while walking and/or standing. However, the conventional cane is only used for support and contains no other features. Specifically, the elderly person is prone to forgetting important medication, bringing an umbrella for rain, bringing a phone to communicate with family, and/or having difficulty seeing in the dark. As such, the elderly person has no ability to readily contact a family member in case of falling due to striking an object in the dark.

Therefore, there is a need for a posture support device with additional functions.

### SUMMARY

The present general inventive concept provides a posture support device with additional functions.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a posture support device, including a main body to support a weight of a user thereon, a control unit disposed within at least a portion of the main body to connect the user to an external mobile device of a third party in response to an emergency, and to display the third party on the control unit, a speaker disposed on at least a portion of the main body to emit at least one sound received from the external mobile device during conversations with the third party, and a medication compartment disposed around at least a portion of the main body to store at least one medication therein.

The main body may include an outer portion, an inner portion slidably disposed within at least a portion of a base of the outer portion to move telescopically in and out of the outer portion, and a storage compartment disposed within at least a portion of the outer portion and the inner portion to store at least one item therein.

The posture support device may further include an umbrella removably disposed within at least a portion of the storage compartment.

The control unit may include a communication unit disposed within at least a portion of the control unit to provide a live feed of the user to the external mobile device, and a sensor disposed within at least a portion of the control unit to alert the third party via the communication unit in response to a sudden change in height of the main body and the weight of the user on the main body.

The posture support device may further include a handle pivotally disposed on at least a portion of a first end of the main body to facilitate gripping thereof.

The posture support device may further include a tripod assembly disposed on at least a portion of a second end of the main body to stabilize the main body.

### BRIEF DESCRIPTION OF THE DRAWINGS

These and/or other features and utilities of the present generally inventive concept will become apparent and more readily appreciated from the following description of the embodiments, taken in conjunction with the accompanying drawings of which:

FIG. 1 illustrates a side perspective view of a posture support device with a handle in a closed position, according to an exemplary embodiment of the present general inventive concept; and

FIG. 2 illustrates a side perspective view of the posture support device with a handle in an opened position, according to an exemplary embodiment of the present general inventive concept.

### DETAILED DESCRIPTION

Various example embodiments (a.k.a., exemplary embodiments) will now be described more fully with reference to the accompanying drawings in which some example embodiments are illustrated. In the figures, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

### LIST OF COMPONENTS

Posture Support Device 100
Main Body 110
Outer Portion 111
Inner Portion 112
Support Member Receiving Grooves 112a
Protruding Members 112b
Storage Compartment 113
Handle 120
Illumination Assembly 130
Flashlight 131
Light Button 132
Umbrella 140
Control Unit 150
Input Unit 151
Display Unit 152
Processing Unit 153
Communication Unit 154
Storage Unit 155
Sensor 156
Speaker 160
Medication Compartment 170
Tripod Assembly 180
Support Members 181
Switch 182
Power Source 190

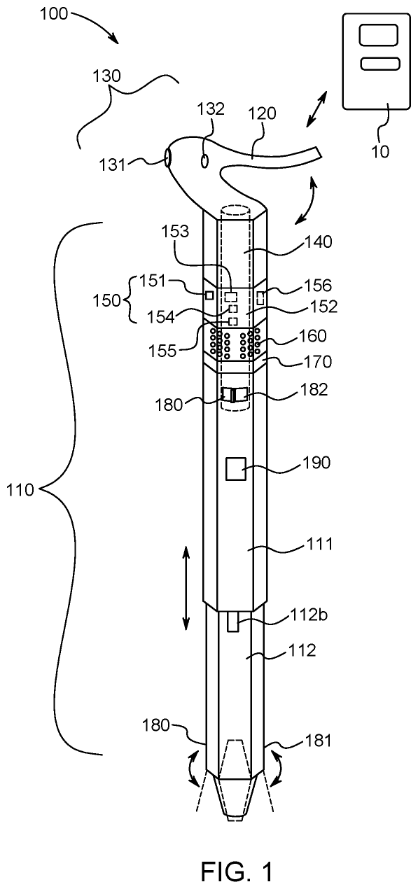

FIG. 1 illustrates a side perspective view of a posture support device 100 with a handle 120 in a closed position, according to an exemplary embodiment of the present general inventive concept.

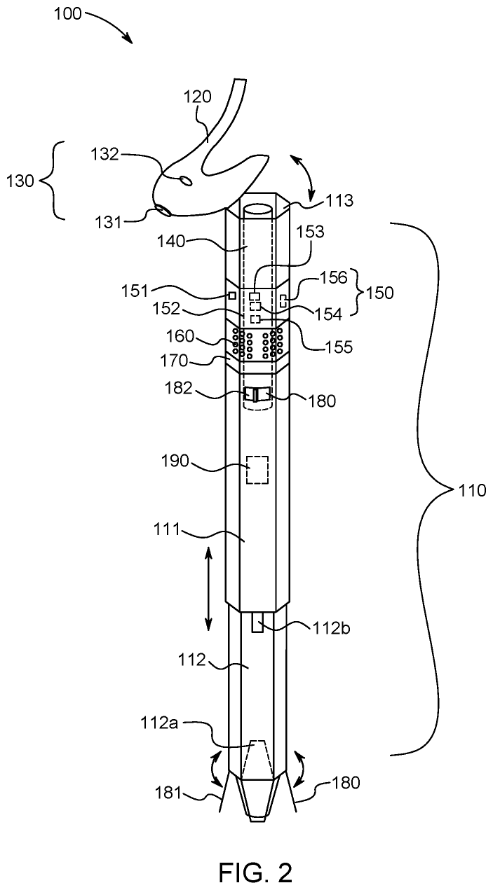

FIG. 2 illustrates a side perspective view of the posture support device with the handle 120 in an opened position, according to an exemplary embodiment of the present general inventive concept.

The posture support device 100 may be constructed from at least one of metal, plastic, wood, glass, ceramic, and rubber, etc., but is not limited thereto.

The posture support device 100 may include a main body 110, a handle 120, an illumination assembly 130, an umbrella 140, a control unit 150, a speaker 160, a medication compartment 170, a tripod assembly 180, and a power source 190, but is not limited thereto.

Referring to FIGS. 1 and 2, the main body 110 is illustrated to have a hexagonal prism shape. However, the main body 110 may be rectangular, circular, cylindrical, conical, pentagonal, heptagonal, octagonal, or any other shape known to one of ordinary skill in the art, but is not limited thereto.

The main body 110 may include an outer portion 111, an inner portion 112, and a storage compartment 113, but is not limited thereto.

The outer portion 111 may have a size greater than the inner portion 112. Moreover, the inner portion 112 may be slidably disposed within at least a portion of a base of the outer portion 111. In other words, the inner portion 112 may move telescopically in and/or out of the outer portion 111.

The outer portion 112 may include a plurality of support member receiving grooves 112a and a plurality of protruding members 112b, but is not limited thereto.

The plurality of support member receiving grooves 112a may be disposed around at least a portion of a first end of the inner portion 112, such that each of the plurality of support member receiving grooves 112a may be disposed a distance away from each other. Also, each of the plurality of support member receiving grooves 112a may be recessed with respect to an outer surface of the inner portion 112.

The plurality of protruding members 112b may be disposed around at least a portion of the outer surface of the inner portion 112, such that each of the plurality of protruding members 112b may be disposed the distance away from each other. Furthermore, the plurality of protruding members 112b may be disposed at multiple positions along a length of the inner portion 112. For example, a first set of the plurality of protruding members 112b may be disposed at a center of the inner portion 112, and a second set of the plurality of protruding members 112b may be disposed at an intermediate point between the center of the inner portion 112 and a second end of the inner portion 112. In other words, the inner portion 112 may be removed at intervals based on each set of the plurality of protruding members 112b.

Each of the plurality of protruding members 112b may protrude away from the outer surface of the inner portion 112 in response to the inner portion 112 being extracted from within the outer portion 111, such that the inner portion 112 may be prevented from moving back within the outer portion 111. Alternatively, the plurality of protruding members 112b may retract within the inner portion 112 in response to a force being applied thereto. In other words, the plurality of protruding members 112b may be pushed and/or pulled back into the inner portion 112, such that the inner portion 112 may move back within the outer portion 111.

The storage compartment 113 may be disposed within at least a portion of the main body 110. The storage compartment 113 may store at least one item therein.

The handle 120 may be removably disposed on at least a portion of a first end of the main body 110. Alternatively, the handle 120 may be pivotally disposed on at least a portion of the first end of the main body 110. As such, the handle 120 may move from closed in a first position to at least partially opened in a second position, such that the storage compartment 113 may be accessed. Conversely, the handle 120 may move from opened in the second position to closed in a first position, such that the storage compartment 113 may not be accessed.

The illumination assembly 130 may include a flashlight 131 and a light button 132, but is not limited thereto.

Referring again to FIGS. 1 and 2, the flashlight 131 is illustrated to be disposed on the handle 120. However, the flashlight 131 may be disposed on any portion of the main body 110.

The flashlight 131 may illuminate a surrounding area in response to depressing the light button 132. Moreover, the flashlight 131 may flash and/or illuminate a first color in response to depressing the light button 132 for a first predetermined length of time and/or a first predetermined number of times. Also, the flashlight 131 may flash and/or illuminate a second color in response to depressing the light button 132 for a second predetermined length of time and/or a second predetermined number of times.

The umbrella 140 may be removably disposed within at least a portion of the storage compartment 113. As such, the umbrella 140 may be extracted while the handle 120 is opened.

The control unit 150 may include an input unit 151, a display unit 152, a processing unit 153, a communication unit 154, a storage unit 155, and a sensor 156, but is not limited thereto.

The input unit 151 may include a keyboard, a touchpad, a mouse, a trackball, a stylus, a voice recognition unit, a visual data reader, a camera, a wireless device reader, and a holographic input unit.

The display unit 152 may include a plasma screen, an LCD screen, a light emitting diode (LED) screen, an organic LED (OLED) screen, a computer monitor, a hologram output unit, a sound outputting unit, or any other type of device that visually or aurally displays data.

Also, the display unit 152 may be combined with the input unit 151 to be a touch-screen.

The processing unit 153 (or central processing unit, CPU) may include electronic circuitry to carry out instructions of a computer program by performing basic arithmetic, logical, control and input/output (I/O) operations specified by the instructions. The processing unit 153 may include an arithmetic logic unit (ALU) that performs arithmetic and logic operations, processor registers that supply operands to the ALU and store the results of ALU operations, and a control unit that fetches instructions from memory and “executes” them by directing the coordinated operations of the ALU, registers and other components. The processing unit 153 may also include a microprocessor and a microcontroller.

The communication unit 154 may include a device capable of wireless or wired communication between other wireless or wired devices via at least one of Wi-Fi, Wi-Fi Direct, infrared (IR) wireless communication, satellite communication, broadcast radio communication, Microwave radio communication, Bluetooth, Bluetooth Low Energy (BLE), Zigbee, near field communication (NFC), and radio frequency (RF) communication, USB, Firewire, and Ethernet.

The storage unit 155 may include a random access memory (RAM), a read-only memory (ROM), a hard disk, a flash drive, a database connected to the Internet, cloud-based storage, Internet-based storage, or any other type of storage unit.

The sensor 156 may include a weight sensor, a height sensor, a global positioning system (GPS), a temperature sensor, or any other type of sensor.

The control unit 150 may access the Internet via the communication unit 154 to allow a user to access a website, and/or may allow a mobile and/or a software application to be executed using the processing unit 153. For ease of description, the mobile and/or the software application will be hereinafter referred to as an app. The app may be downloaded from the Internet to be stored on the storage unit 155.

The control unit 150 may receive input by the user from the input unit 151 to enter contact information of an external mobile device 10, used by a third party, into the app, such as a family member, a friend, a doctor, and/or an emergency response unit (e.g., police, fire department, rescue). Also, the control unit 150 may use the app to connect the user to the third party via the communication unit 154 in response to an emergency. Specifically, the sensor 156 may detect a weight of the user upon the main body 110, a temperature of the user on the main body 110, a position of the user, and/or a height of the user. As such, the sensor 156 may send an alert signal to the processing unit 153 in response to a change in the weight on the main body 110, the temperature of the user on the main body 110, the position of the user, and/or the height of the user while using the main body 110. Also, the sensor 156 may be programmed to send the alert only if there is a sudden change, which would indicate a fast change in height of the main body 110 and/or the weight on the main body 110 that would indicate the user likely has fallen. Any such data may be stored on the storage unit 155.

Additionally, the processing unit 153 may gather data from the storage unit 155 to send via the communication unit 154 to the external mobile device 10. Accordingly, the external mobile device 10 may be alerted that the user has likely been injured and needs assistance.

The processing unit 153 may also send a call and/or a message signal to send a request for assistance via the communication unit 154. As such, the communication unit 154 may interface with the external mobile device 10 to begin a phone call, a video call, and/or a messaging app. Alternatively, the processing unit 153 may use the input unit 151 to provide live feeds to the external mobile device 10, such that the external mobile device 10 receives constant video streams of the user.

The processing unit 153 may also be programmed via the app to set up reminders, such as medication reminders, medical and/or any other appointments, allergies, and/or weather updates. As such, the processing unit 153 may retrieve the reminders on a daily basis, such that the user does not forget important information. Furthermore, the reminders may be audio reminders and/or visual reminders on the display unit 152.

The speaker 160 may be disposed on at least a portion of the main body 110. The speaker 160 may emit a sound in response to commands received from the control unit 150. For example, the speaker 160 may emit sounds from conversations during the phone call and/or the video call to the external mobile device 10, as well as the reminders set as audio alerts.

The medication compartment 170 may include a door, but is not limited thereto. The medication compartment 170 may be disposed around at least a portion of the main body 110. The medication compartment 170 may store at least one medication therein. The door of the medication compartment 170 may be opened to allow access to the at least one medication, and closed to prevent access to the at least one medication.

The tripod assembly 180 may include a plurality of support members 181 and a switch 182, but is not limited thereto.

Each of the plurality of support members 181 may be pivotally disposed on at least a portion of the first end of the inner portion 112. Moreover, each of the plurality of support members 181 may be distanced from each other around the first end of the inner portion 112. Each of the plurality of support member receiving grooves 112a may receive at least one of the plurality of support members 181 therein.

More specifically, each of the plurality of support members 181 may pivot from retracted within at least one of the plurality of support member receiving grooves 112a in a first vertical position to at least partially extended away from each of the plurality of support member receiving grooves 112a in a second vertical position. Conversely, each of the plurality of support members 181 may pivot from extended in the second position to retracted within at least one of the plurality of support member receiving grooves 112a in the first position.

Each of the plurality of support members 181 may pivot in response to movement of the switch 181 from retracted to extended, and extended to retracted. As such, the plurality of support members 181 may stabilize the main body 110 while in the extended position.

The power source 190 may include a battery and a solar cell, but is not limited thereto.

The power source 190 may be disposed within at least a portion of the main body 110. The power source may provide power to the illumination assembly 130, the control unit 150, the speaker 160, and/or the tripod assembly 180.

Therefore, the posture support device 100 may provide additional features to aid the user after suffering a fall. In particular, the posture support device 100 may alert a friend or family member if the user should fall. Additionally, the posture support device 100 may remind the user of important events without having to rely on the user's memory.

The present general inventive concept may include a posture support device 100, including a main body 110 to support a weight of a user thereon, a control unit 150 disposed within at least a portion of the main body 110 to connect the user to an external mobile device 10 of a third party in response to an emergency, and to display the third party on the control unit 150, a speaker 160 disposed on at least a portion of the main body 110 to emit at least one sound received from the external mobile device 10 during conversations with the third party, and a medication compartment 170 disposed around at least a portion of the main body 110 to store at least one medication therein.

The main body 110 may include an outer portion 111, an inner portion 112 slidably disposed within at least a portion of a base of the outer portion 111 to move telescopically in and out of the outer portion 111, and a storage compartment 113 disposed within at least a portion of the outer portion 111 and the inner portion 112 to store at least one item therein.

The posture support device 100 may further include an umbrella 140 removably disposed within at least a portion of the storage compartment 113.

The control unit 150 may include a communication unit 154 disposed within at least a portion of the control unit 150 to provide a live feed of the user to the external mobile device 10, and a sensor 156 disposed within at least a portion of the control unit 150 to alert the third party via the communication unit 154 in response to a sudden change in height of the main body 110 and the weight of the user on the main body 110.

The posture support device 100 may further include a handle 120 pivotally disposed on at least a portion of a first end of the main body 110 to facilitate gripping thereof.

The posture support device 100 may further include a tripod assembly 180 disposed on at least a portion of a second end of the main body 110 to stabilize the main body 110.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A posture support device, comprising:
a main body to support a weight of a user thereon;
a control unit disposed within at least a portion of the main body to connect the user to an external mobile device of a third party in response to an emergency, and to display the third party on the control unit;
a speaker disposed on at least a portion of the main body to emit at least one sound received from the external mobile device during conversations with the third party; and
a medication compartment disposed around at least a portion of the main body to store at least one medication therein.

2. The posture support device of claim 1, wherein the main body comprises:
an outer portion;
an inner portion slidably disposed within at least a portion of a base of the outer portion to move telescopically in and out of the outer portion; and
a storage compartment disposed within at least a portion of the outer portion and the inner portion to store at least one item therein.

3. The posture support device of claim 2, further comprising:
an umbrella removably disposed within at least a portion of the storage compartment.

4. The posture support device of claim 1, wherein the control unit comprises:
a communication unit disposed within at least a portion of the control unit to provide a live feed of the user to the external mobile device; and
a sensor disposed within at least a portion of the control unit to alert the third party via the communication unit in response to a sudden change in height of the main body and the weight of the user on the main body.

5. The posture support device of claim 1, further comprising:
a handle pivotally disposed on at least a portion of a first end of the main body to facilitate gripping thereof.

6. The posture support device of claim 5, further comprising:
a tripod assembly disposed on at least a portion of a second end of the main body to stabilize the main body.
